(19) United States
(12) Patent Application Publication
(10) Pub. No.: US 2025/0163488 A1
(43) Pub. Date: May 22, 2025

(54) CONJUGATION METHOD INVOLVING A TRANSGLUTAMINASE AT THE FC REGION COMPRISING A TRIMMED N-GLYCAN

(71) Applicant: ADC THERAPEUTICS SA, Epalinges (CH)

(72) Inventors: Matthew BIRD, London (GB); Patricius Hendrikus Cornelis VAN BERKEL, London (GB)

(21) Appl. No.: 18/840,324

(22) PCT Filed: Feb. 22, 2023
(86) PCT No.: PCT/EP2023/054469

§ 371 Date: Aug. 21, 2024

(30) Foreign Application Priority Data
Feb. 22, 2022 (EP) 22158087.1
Jul. 4, 2022 (EP) 22182782.7

(51) Int. Cl. C12P 21/00; A61K 47/68; C12N 9/10; C12N 9/24

(52) U.S. Cl. CPC C12P 21/005; A61K 47/6889; C12N 9/1044; C12N 9/2402; C12Y 203/02013; C12Y 302/01096

(57) ABSTRACT

Described herein are methods for conjugating moieties of interest, such as pharmaceutically active molecules, to targeting molecules such as antibodies using transglutaminase-based processes.

### FIELD OF THE INVENTION

The present disclosure relates to methods for conjugating moieties of interest, such as pharmaceutically active molecules, to targeting molecules such as antibodies.

### BACKGROUND TO THE INVENTION

A variety of methods are known for the attachment of pharmaceutical actives to antibodies and related molecules. For example, drug linkers have been attached to native or engineered cysteine residues in immunoglobulins using a maleimide group. Other approaches have been based on using the N-linked glycosylation site, N297, which in some approaches is remodeled to trim back the glycans to a core GlcNAc moiety and then reacting this with a UDP-sugar in the presence of a GalT enzyme, the UDP-sugar further include a reactive azide moiety which allows for the subsequent addition of a drug-linker using click chemistry. Another approach has been to use endogenous or engineered glutamine residues together with microbial transglutaminase (mTGase).

### SUMMARY OF THE INVENTION

Prior approaches to using mTGase to attach at the endogenous glutamine residue at position Q295 (Kabat EU numbering) in the Fc region of immunoglobulins, have been based on using PNGaseF which removes all of the N-linked glycans at neighbouring position N297 to avoid interference with the TGase reaction. This leaves a negatively charged aspartic acid residue at D297 and therefore modified approaches involve mutating D297 to remove the negative charge (and also the need to remove the N-glycans). We have now found that using a different enzymatic treatment of the N-glycans, based on EndoS, to leave the core GlcNAc moiety intact, allows for highly efficient mTGase reactions at Q295. Further, the core GlcNAc moiety at N297 is then available for a second conjugation, which we have surprisingly found to be highly efficient even though a drug-linker is already present at the adjacent Q295.

Accordingly, in a first aspect the present disclosure relates to a method for conjugating a moiety of interest to an Fc-containing polypeptide (e.g. an antibody), which method comprises:
(i) providing an Fc-containing polypeptide with N-linked glycosylation at a site present in the Fc region of the polypeptide, adjacent a site comprising a glutamine residue;
(ii) treating said polypeptide with an enzyme, such as EndoS, to trim back the N-linked glycans at the site of N-linked glycosylation to an optionally fucosylated, core N-acetylglucosamine (GlcNAc) moiety; and
(iii)(1) reacting, in the presence of a transglutaminase (TGase), the polypeptide obtained in step (ii) with a moiety of interest comprising an acceptor moiety which is a substrate for the TGase, so that the moiety of interest is attached to the polypeptide via the glutamine residue.

In a second related aspect, the present disclosure also relates to a method for conjugating a moiety of interest to an Fc-containing polypeptide (e.g. an antibody), which method comprises:
(i) providing an Fc-containing polypeptide with N-linked glycosylation at a site present in the Fc region of the polypeptide, adjacent a site comprising a glutamine residue;
(ii) treating said polypeptide with an enzyme, such as EndoS, to trim back the N-linked glycans at the site of N-linked glycosylation to an optionally fucosylated, core N-acetylglucosamine (GlcNAc) moiety; and
(iii)(2) reacting, in the presence of a transglutaminase (TGase), the polypeptide obtained in step (ii) with an acceptor moiety which is a substrate for the TGase and which acceptor moiety further comprises a reactive group (R) which enables the subsequent attachment of the moiety of interest via a reaction between R and a complementary reactive group (R′) attached to the moiety of interest, such that the acceptor moiety is attached to the polypeptide via the glutamine residue; and
(iv) reacting the polypeptide conjugate formed in step (iii)(2) with the moiety of interest attached to R′ under conditions such that R reacts with R′ to form R—R′.

In one embodiment of the above aspects, the acceptor moiety is selected from an amine, an aminooxy, a hydrazido, a hydrazino and aryl derivatives thereof.

In one embodiment, the acceptor moiety is of formula Ac-Sp-R where Ac is selected from an amine, an aminooxy, a hydrazido, a hydrazino and aryl derivatives thereof, Sp is absent or a spacer moiety; and R is a reactive group, e.g. an azide.

In one embodiment, the N-linked glycosylation site is at position 297 according to the Kabat numbering system, or an equivalent position and/or the site comprising a glutamine residue is at position 295 according to the Kabat numbering system, or an equivalent position.

In one embodiment, the moiety of interest comprises a drug-linker.

The present disclosure also relates to a second aspect wherein in the methods of the first aspect above a second moiety of interest is conjugated to the Fc-containing polypeptide, which method further comprises the following step:
(v) reacting, in the presence of a galactosyl transferase (GalT), the polypeptide obtained in step (iii)(1), step (iii)(2) or step (iv) with a sugar derivative attached to the second moiety of interest, so that the sugar derivative is attached to the polypeptide via the core N-acetylglucosamine (GlcNAc) moiety.

In a related aspect, a second moiety of interest is conjugated to the Fc-containing polypeptide, which method further comprises the following steps:
(v) reacting, in the presence of a galactosyl transferase (GalT), the polypeptide obtained in step (iii))(1), step (iii)(2) or step (iv) with a sugar derivative attached to a reactive group (R) which enables the subsequent attachment of the moiety of interest via a reaction between R and a complementary reactive group (R′) attached to the moiety of interest, so that the sugar derivative is attached to the polypeptide via the core N-acetylglucosamine (GlcNAc) moiety; and
(vi) reacting the polypeptide conjugate formed in step (v) with the moiety of interest attached to R′ under conditions such that R reacts with R′ to form R—R′.

The present disclosure, in a third aspect, relates to an Fc-containing polypeptide conjugate, which comprises:
(i) an asparagine residue present in the Fc region of the polypeptide with an optionally fucosylated, GlcNAc N glycan core lacking any additional endogenous glycan residues; and
(ii) (Q)-NH-L-D, or a pharmaceutically acceptable salt or solvate thereof; wherein Q is a glutamine residue present in the Fc region of the polypeptide; L is a linker, which optionally comprises a conditionally-cleavable moiety; and D is a drug moiety;
wherein the asparagine residue is adjacent the glutamine residue.

In one embodiment, in the Fc-containing polypeptide conjugate, L comprises L₁-R—R′-L₂; wherein L₁ is absent or a spacer moiety, R—R′ is formed by the reaction of a reactive group R and a complementary reactive group R′; and L₂ is a linker or spacer moiety to which D is attached and which optionally comprises a conditionally-cleavable moiety.

The present disclosure, in a fourth aspect relates to an Fc-containing polypeptide conjugate, which comprises:
(i) (Q)-NH-LA-D1, or a pharmaceutically acceptable salt or solvate thereof; wherein Q is a glutamine residue present in the Fc region of the polypeptide; LA is a linker; and D is a drug moiety;
(ii) (N)-G-S—R₂—R₂′-LB-D2, or a pharmaceutically acceptable salt or solvate thereof; where N is an asparagine residue present in the Fc region of the polypeptide; G is an optionally fucosylated, GlcNAc N glycan core; S is a sugar derivative; R₂—R₂′ is formed by the reaction of a reactive group R₂ and a complementary reactive group R₂′; LB is a linker, which optionally comprises a conditionally-cleavable moiety; and D1 and D2 are each independently a drug moiety; and
wherein the asparagine residue is adjacent the glutamine residue.

In one embodiment, in the Fc-containing polypeptide conjugate LA comprises LA₁-R₁—R₁′-LA₂; wherein LA₁ is absent or a spacer moiety, R₁—R₁′ is formed by the reaction of a reactive group R₁ and a complementary reactive group R₁′; and LA₂ is a linker or spacer moiety to which D1 is attached and which optionally comprise a conditionally-cleavable moiety.

In an embodiment of the third or fourth aspects, the Fc-containing polypeptide conjugate is an lgG, such as IgG which binds specifically to a tumour antigen.

In an embodiment of the third or fourth aspects, the drug is a cytotoxin.

In an embodiment of the third or fourth aspects, the asparagine residue is at position 297 according to the Kabat numbering system, or an equivalent position and/or the glutamine residue is at position 295 according to the Kabat numbering system, or an equivalent position.

The present disclosure also relates to an Fc-containing polypeptide conjugate obtained or obtainable by the process of the first or second aspects described above.

In a fifth aspect, the present disclosure relates to a pharmaceutical composition comprising an Fc-containing polypeptide conjugate as described herein together with one or more pharmaceutically acceptable carriers or diluents, such as a buffering agent, a sugar and/or a detergent.

### DETAILED DESCRIPTION

### Antibodies

Antibodies for use in the processes described herein comprise an N-glycosylated asparagine residue, such as the wild-type N297 in the heavy chain of an immunoglobulin, or the equivalent position. N-glycosylation sites can also be engineered into the antibody, for example based on the consensus sequence (sequon) Asn-Xaa-Ser/Thr where Xaa is any amino acid except proline (see Reslan et al., 2020, Int. J. Biol. Macromol 158:189-196; Cruz et al., 2021 Pharmaceutics 13 (11): 1747; Bañó-Polo et al., 2011, Protein Sci. 20 (1): 179-186).

The N-glycosylated asparagine residue is flanked by a glutamine residue that is a substrate for microbial transglutaminase. For example, immunoglobulins with an N297 glycosylation site typically have a glutamine residue at position 295 (i.e. 2 residues from the N297 residue). “Flanked” means that the glutamine residue is within 2, 3 or 4 amino acids of the N-glycosylated asparagine residue, either side (i.e. in the N-terminal direction or the C-terminal direction). In one embodiment the glutamine residue is at least 2 amino acids from the N-glycosylated asparagine residue, such as +/−2 amino acids.

The glutamine may be naturally occurring/wild type, such as a glutamine residue at position 295, or its equivalent, in an immunoglobulin molecule, or it may be engineered into the antibody amino acid sequence.

The term antibodies includes full length immunoglobulins, e.g. IgGs, as well as fragments thereof that comprise an antigen binding site. In one embodiment, the antibody comprises an Fc domain or a fragment thereof comprising a CH2 domain.

In one embodiment, the N-glycosylation site or sites and flanking glutamine resides are present in a constant region of the antibody, such as the Fc domain or CH2 domain.

Antibodies for use in the processes described herein, and resulting from such processes, may bind to any antigen of interest. In one embodiment the antigen is present on the surface of a cell, such as a cell in the body of a human or animal. Such cells/antigens may be associated with a disease or disorder, for example a proliferative disease. The expression of the antigen may be up-regulated in the cell of interest. In one embodiment the antigen is a tumour antigen. In another embodiment the antigen is a cell-surface molecule found in immune cells, such as regulatory T-cells. Specific cell-surface proteins/tumour antigens include CD19, CD22, CD25, AXL, KAAG1, DLK-1 and PSMA.

### Antibody Re-Modeling

In one aspect of the present invention, the antibody is treated to trim N-glycans, such as the N-glycans at N297, back to core glycans, such as an optionally fucosylated, core N-acetylglucosamine (GlcNAc) moiety. This can be performed by incubating the antibody with an enzyme or enzymes that can remove the majority of the glycans. Previously, N-glycan removal has been carried out using PNGaseF. However, this results in removal of all glycan residues and converts the asparagine to a negatively charged aspartic acid residue. Instead, the process of the present disclosure uses a suitable enzyme to trim back the N-linked glycans at the site of N-linked glycosylation to an optionally fucosylated, core N-acetylglucosamine (GlcNAc) moiety.

Depending on the nature of the glycan, a suitable endoglycosidase may be selected. The endoglycosidase is preferably selected from the group consisting of Endo S, Endo A, EfEndo18A, Endo F (e.g. F1, F2 or F3), Endo M, Endo D and Endo H enzymes and/or a combination thereof, the selection of which depends on the nature of the glycan. In a further preferred embodiment, the endoglycosidase is Endo S, Endo S49, Endo F or a combination thereof. Combination endoglycosidases are disclosed in U.S. Pat. No. 10,858,41, which include two different Endo S enzymes as a fusion protein. The advantage of using combinations is that a greater number of glycoforms can be recognized and trimmed in a single reaction.

EndoS is an endoglycosidase that has been isolated from the human pathogen Streptococcus pyogenes. Different forms of EndoS, such as EndoS (Collin et al., 2001. EMBO J. 20:3046-3055) and EndoS2 (Sjögren et al., 2013. Biochem J. 455:107-118), have been shown to hydrolyze N-linked glycans of human immunoglobulin G, with different glycoform selectivity (Sjögen et al., 2015. Glycobiology 25 (10): 1053-1063). EndoS and EndoS2 are available from Genovis AB (IgGZERO™, GlycINATOR™).

The antibody of interest is incubated with one or more enzymes under suitable conditions to effect removal of all or a substantial portion of the N-glycans to leave the core N-acetylglucosamine (GlcNAc) moiety. A suitable method is described in the examples, as well as by Sjögen et al., 2015, ibid.

An antibody glycan having a core N-acetylglucosamine is herein defined as an antibody comprising a glycan that is bonded to the antibody via C1 of an, optionally fucosylated, GlcNAc (the core-GlcNAc).

### Addition of Moiety of Interest Using TGase

Moieties of interest for conjugation to the Fc-containing polypeptide of interest includes pharmaceutically active substances/drugs, diagnostic reagents and detectable labels. In one embodiment, the active moiety is covalently bonded to a linker or spacer construct.

In the first step of the process, conjugation is effected at a glutamine residue of interest that is adjacent an N-glycosylation site, using a transglutaminase.

Microbial transglutaminase (mTG) catalyzes a transamidation reaction between certain surface-exposed glutamine (Gln) and lysine (Lys) residues on protein substrates, crosslinking the two via an isopeptide bond. The catalytic mechanism of mTG involves a nucleophilic attack of the target acyl donor, Gln, by the enzyme's active site cysteine to form a thioester intermediate. Subsequent nucleophilic attack of the thioester by an amine substrate serving as the acyl acceptor results in the formation of the transamidated product.

The most commonly used mTGase is the 38-kDa enzyme derived from Streptomyces mobaraensis. Another transglutaminase found in bacteria is from Bacillus subtilisin (See U.S. Pat. No. 5,731,183). Various variants of mTGase have been developed such as the mTGases disclosed in WO2017/059158 and WO2017/059160 (Merck) which have for example an amino acid substitution selected from the group consisting of (A) E300A, (B) 1240A and P241A, (C) E249Q, and (D) E300A and Y302A.

The re-modeled antibody can therefore be conjugated to a moiety of interest using a transglutaminase (such as mTGase)—the moiety of interest comprising/being linked to an acyl acceptor which forms a substrate for a mTGase and the glutamine residue of interest in the re-modeled antibody providing the acyl donor for the transamidation reaction. The acyl acceptor commonly used in this reaction is a primary amine. However recent work has shown that other acyl acceptors comprising a terminal-NH group can be used, namely alpha-effect nucleophiles such as hydrazines, hydrazides, and alkoxyamines (Chio et al., 2020. Angew Chem Int Ed Engl 59 (33): 13814-13820). A skilled person will appreciate that the term ‘alpha effect’ refers to the increased nucleophilicity of an atom due to the presence of an adjacent (alpha) atom with lone pair electrons; in this context the increased nucleophilicity of the nitrogen atom of the terminal-NH of the acyl acceptor. In one embodiment the acyl acceptor is selected from an amine, such as a primary amine; a hydrazine; a hydrazide; an alkoxyamine or aryloxyamine. In a further embodiment, the amine, hydrazine, hydrazide or oxyamine is an aryl derivative, in particular an optionally substituted phenyl or optionally substituted benzyl derivative e.g. optionally substituted phenylhydrazine In one embodiment, the acyl acceptor is a hydrazine or a hydrazide. In one embodiment, the acyl acceptor is a hydrazide or optionally substituted aryl hydrazine derivative. In a further embodiment, the acyl acceptor is an optionally substituted aryl hydrazine derivative or an optionally substituted aryl hydrazide derivative. In a yet further embodiment, the acyl acceptor is an optionally substituted phenylhydrazine or optionally substituted phenylhydrazide derivative.

In one embodiment, the acyl acceptor comprises an optionally substituted moiety selected from:

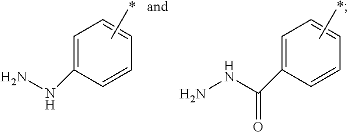

in particular

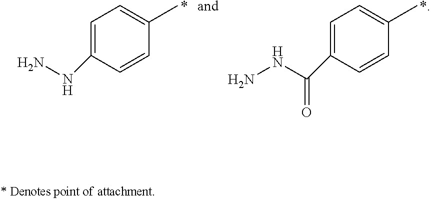

Alternatively, the acyl acceptor comprises an optionally substituted moiety selected from:

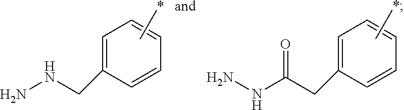

in particular

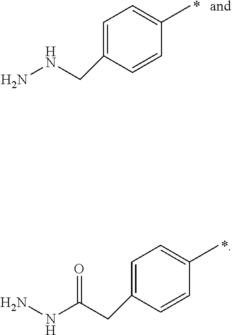

In a further embodiment, the acyl acceptor comprises an optionally substituted moiety selected from

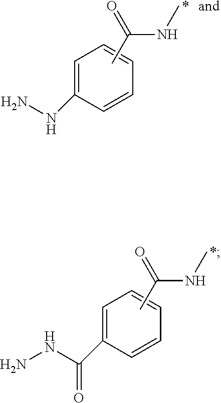

in particular

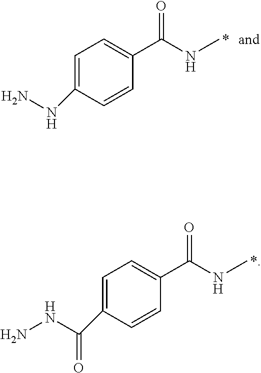

In a further alternative embodiment, the acyl acceptor comprises a moiety selected from

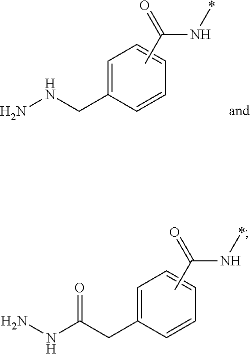

in particular

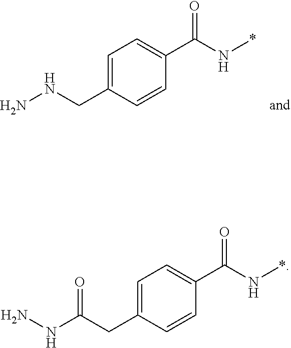

In one embodiment, the aryl derivative may be multiply substituted such that the acyl acceptor is branched with multiple points of attachment.

In one embodiment, the acyl acceptor comprises an optionally substituted moiety selected from:

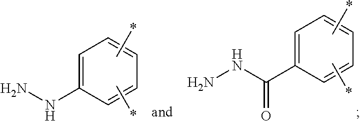

in particular

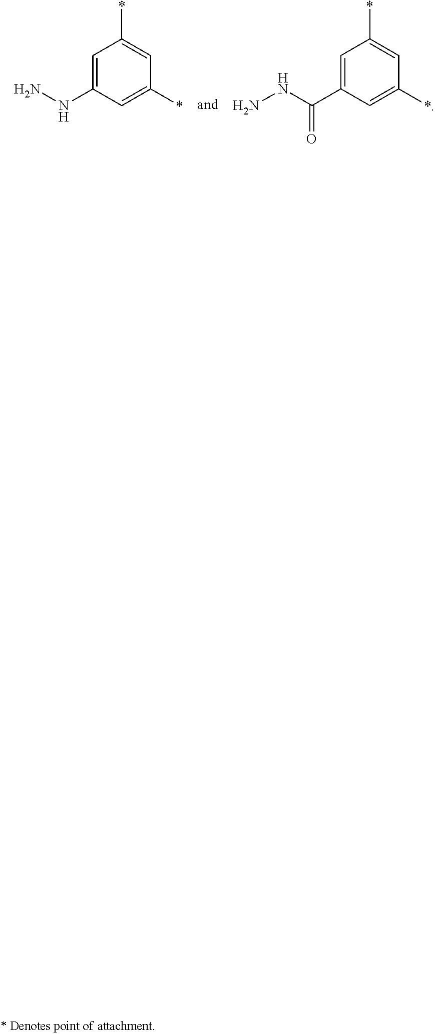

Alternatively, the acyl acceptor comprises an optionally substituted moiety selected from:

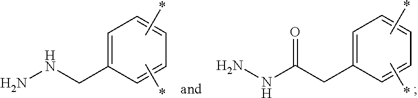

in particular

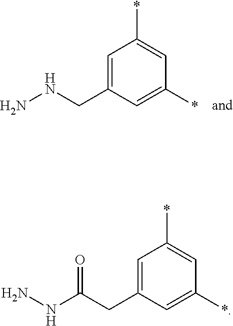

Specific examples include:

| | | |
|---|---|---|
| 1 | amine-PEG(3u)-azide | 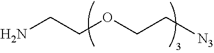 |
| 2 | aminooxy-PEG(3u)-azide | 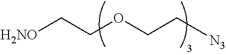 |
| 3 | hydrazido-PEG(4u)-azide | 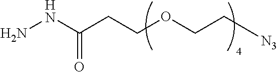 |
| 4 | hydrazino-PEG(4u)-azide | |
| 5 | 4-aminobenzamide- | |
| | PEG(4u)-azide | |
| 6 | 4-aminooxybenzamide- | |
| | PEG(4u)-azide | |
| 7 | 4-hydrazidobenzamide- | |
| | PEG(4u)-azide | |
| 8 | 4-hydrazinobenzamide- | |
| | PEG(4u)-azide | |
| 9 | 4-aminomethylbenzamide- | |
| | PEG(4u)-azide | |
| 10 | 4-aminooxymethylbenzamide- | |
| | PEG(4u)-azide | |
| 11 | 4-hydrazidomethylbenzamide- PEG(4u)-azide | 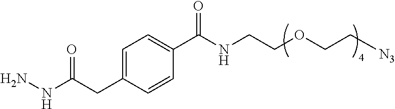 |
| 12 | 4-hydrazinomethylbenzamide- PEG(4u)-azide | 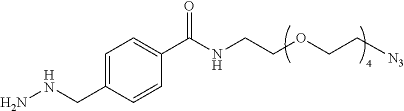 |

In one embodiment, the acyl acceptor is selected from Examples 1 to 12, in particular Examples 3, 4, 7, 8, 11 and 12.

The azide group in examples 1 to 12 can be substituted with other reactive groups as described below for two step reactions, such as BCN and DBCO. The azide group can also be replaced with other linker components.

As used herein, the term “amine” refers to derivatives of ammonia, wherein one or more hydrogen atoms have been replaced by a substituent such as an aliphatic (e.g. alkyl or heteroalkyl) or aromatic (e.g. aryl or heteroaryl) group. A primary amine has only one of three hydrogen atoms in ammonia is replaced by a substituent.

As used herein, the term “hydrazines” refers to derivatives of hydrazine (H₂N—NH₂), in which one of the hydrogen atoms has been replaced by a substituent such as an aliphatic (e.g. alkyl or heteroalkyl) or aromatic (e.g. aryl or heteroaryl) group.

As used herein, the term “hydrazides” refers to derivatives of the inorganic hydrazine (H₂N—NH₂), in which one of the hydrogen atoms has been replaced by a substituent such as an acyl, a sulfonyl or a phosphoryl group, in particular an acyl group.

As used herein, the term “alkyl” refers to a fully saturated branched or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, the term “alkylene” refers to divalent alkyl group as defined herein above having 1 to 20 carbon atoms. It comprises 1 to 20 carbon atoms, Unless otherwise provided, alkylene refers to moieties having 1 to 16 carbon atoms, 1 to 10 carbon atoms, 1 to 7 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like.

As used herein, the term “alkynyl” refers to a branched or unbranched hydrocarbon moiety comprising one triple bond having up to 20 carbon atoms. Unless otherwise provided, alkynyl refers to hydrocarbon moieties having 2 to 16 carbon atoms, 2 to 10 carbon atoms, 2 to 7 carbon atoms, or 2 to 4 carbon atoms. Representative examples of alkyl include, but are not limited to, ethynyl, propynyl, butynyl, pentynyl, hexynyl, heptynyl, octynyl, nonynyl, decynyl and the like.

The term “heteroalkyl” is given its ordinary meaning in the art and refers to alkyl groups as described herein in which one or more carbon atoms is replaced by a heteroatom (e.g., oxygen, nitrogen, sulfur).

The term “aryl” refers to an aromatic hydrocarbon group having 6-20 carbon atoms in the ring portion. Typically, aryl is monocyclic, bicyclic or tricyclic aryl having 6-20 carbon atoms.

Furthermore, the term “aryl” as used herein, refers to an aromatic substituent which can be a single aromatic ring, or multiple aromatic rings that are fused together.

Non-limiting examples include phenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S—, nitro, cyano, carboxy, alkyl-O—C(O)—, carbamoyl, alkyl-S(O)—, sulfonyl, sulfonamido, phenyl, and heterocyclyl.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have about 1-7, more preferably about 1-4 carbons.

As used herein, the term “aryloxy” refers to both an —O-aryl and an —O-heteroaryl group, wherein aryl and heteroaryl are defined herein.

As used herein, the term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring system (e.g., 5-7 membered monocycle or an 8-10 membered bicycle) or a 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2, 3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3, 5-, or 8-pyrazino[2,3-d]pyridazinyl, 2-, 3-, 5-, or 6-imidazo[2,1-b]thiazolyl, 1-, 3, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl.

A heteroaryl group may be substituted with 1 to 5 substituents independently selected from the group consisting of the following:
(a) alkyl;
(b) hydroxy (or protected hydroxy);
(c) halo;
(d) oxo, i.e., ═O;
(e) amino, alkylamino or dialkylamino;
(f) alkoxy;
(g) cycloalkyl;
(h) carboxyl;
(i) heterocyclooxy, wherein heterocyclooxy denotes a heterocyclic group bonded through an oxygen bridge;
(j) alkyl-O—C(O)—;
(k) mercapto;
(l) nitro;
(m) cyano;
(n) sulfamoyl or sulfonamido;
(o) aryl;
(p) alkyl-C(O)—O—;
(q) aryl-C(O)—O—;
(r) aryl-S—;
(s) aryloxy;
(t) alkyl-S—;
(u) formyl, i.e., HC(O)—;
(v) carbamoyl;
(w) aryl-alkyl-; and
(x) aryl substituted with alkyl, cycloalkyl, alkoxy, hydroxy, amino, alkyl-C(O)—NH—, alkylamino, dialkylamino or halogen.

As used herein, the term “acyl” refers to a substituted carbonyl group wherein the carbon atom is substituted by a substituent such as an aliphatic (e.g. alkyl or heteroalkyl) or aromatic (e.g. aryl or heteroaryl) group.

As used herein, the term “halogen” or “halo” refers to fluoro, chloro, bromo, and iodo.

As used herein, the term “optionally substituted” unless otherwise specified refers to a group that is unsubstituted or is substituted by one or more, typically 1, 2, 3 or 4, suitable non-hydrogen substituents, each of which is independently selected from the group consisting of:
(a) alkyl;
(b) hydroxy (or protected hydroxy);
(c) halo;
(d) oxo, i.e., ═O;
(e) amino, alkylamino or dialkylamino;
(f) alkoxy;
(g) cycloalkyl;
(h) carboxyl;
(i) heterocyclooxy, wherein heterocyclooxy denotes a heterocyclic group bonded through an oxygen bridge;
(j) alkyl-O—C(O)—;
(k) mercapto;
(l) nitro;
(m) cyano;
(n) sulfamoyl or sulfonamido;
(o) aryl;
(p) alkyl-C(O)—O—;
(q) aryl-C(O)—O—;
(r) aryl-S—;
(s) aryloxy;
(t) alkyl-S—;
(u) formyl, i.e., HC(O)—;
(v) carbamoyl;
(w) aryl-alkyl-; and
(x) aryl substituted with alkyl, cycloalkyl, alkoxy, hydroxy, amino, alkyl-C(O)—NH—, alkylamino, dialkylamino or halogen.

The acyl acceptor may comprise/be linked to a spacer moiety, such as polyethylene glycol, e.g. polyethylene glycol with from 1 to 50 ethylene glycol units, such as 1 to 30 ethylene glycol units. Suitable spacer moieties may include, but are not limited to, a substituted or unsubstituted alkylene or heteroalkylene chain, the length of the carbon chain (n) being, for example, an integer selected from 2 to 20.

The acyl acceptor may also comprise/be linked to other components of a drug-linker construct. A wide variety of linker technologies are available in the art to link drugs such as cytotoxins to cell binding agents. Linkers can incorporate various different moieties to assist with antibody-drug conjugate stability and determine drug release characteristics. For example the linker may include a cleavable moiety, such as one that is cleavable by cathepsin B (e.g. Valine-Alanine or Valine-Citrulline). Another strategy is to use a pH-sensitive linker whereby the lower pH of the endosome and lysosome compartments the hydrolysis of an acid-labile group within the linker, such as a hydrazone, carbonate, silyl ether or silanol (e.g. —Si(Me₂)-O—Si(Me₂)- see WO2016/183359). Alternative a linker may be non-cleavable, which can avoid or reduce off-target effects and improve plasma stability during circulation.

In another embodiment the linker may include a spacer moiety such as polyethylene glycol e.g. linked to the acyl acceptor or otherwise, and/or a substituted or unsubstituted alkylene or heteroalkylene chain, the length of the carbon chain (n) being an integer selected from 2 to 20. A spacer moiety may also be a hydrophilic spacer such as —NH—SO₂—NH— to increase the overall hydrophilicity of the resulting conjugate (taking into account that many drugs of interest can be quite hydrophobic).

The linker may also comprise an self-immolative group linked to the drug moiety such that the drug can be released without any linker groups remaining. Examples of such groups are p-aminobenzyl alcohol (PABA) and p-amino benzyloxycarbonyl (PABC). Self-immolation is the spontaneous and irreversible fragmentation of a multicomponent compound into small molecules through a cascade of cyclisation or elimination reactions. In this context a self-immolative group is a moiety that undergoes self-immolation and acts as a linker for temporarily connecting a cleavable protecting group (designated as the ‘trigger’) to a drug moiety. The self-immolative group maintains space between the trigger and drug moiety and enhances the entropic driving force that promotes complete and traceless deprotection.

Linkers include both linear and branched configurations. Branched linkers enable multiple active agents to be attached via one conjugation point on the antibody. Various branched linkers are described in WO2016/183359. Branched linkers include branched PEG linkers such as branched PEG azide and branched PEG maleimide.

The drug moiety (D) can be any suitable pharmaceutically active substance that it is desired to deliver to a target cell. Examples include cytotoxins such as taxanes, anthracyclines, camptothecins, epothilones, mytomycins, combretastatins, vinca alkaloids, nitrogen mustards, maytansinoids, calicheamycins, duocarmycins, tubulysines, amanitins/amatoxins, dolastatins, auristatins, enediynes, pyrrolobenzodiazepines, ecteinascidins and ethylenimines. Other examples include immunostimulatory molecules such as TLR agonists (e.g. TLR7, TLR8 and TLR9 agonists) and STING agonists. In one embodiment where two or more drug moieties are present, at least one drug moiety has a first mode of action and at least one drug moiety has a second, different, mode of action. For example at least one is an immunostimulatory molecule and at least one is a cytotoxin.

The drug-linker can be attached to the antibody in one or more steps. For example, in a one step process, the drug-linker with an acyl acceptor moiety is reacted with the antibody of interest with mTGase.

In one embodiment, the resulting molecule comprises the following formula: (Q)-NH-L-D; wherein Q is a glutamine residue present in the Fc region of the polypeptide; NH is derived from the acyl acceptor; L is a linker and D is a drug moiety.

In particular embodiments, taking into account the relevant chemistry of the acyl acceptors, the resulting molecules may comprise any one of the following formulae:

(Q)-NH-L-D amine;

(Q)-NH-Ar-L-D aryl amine;

(Q)-NH—O-L-D aminooxy;

(Q)-NH—O-Ar-L-D aryl aminooxy;

(Q)-NH—NH—C(O)-L-D hydrazide;

(Q)-NH—NH—C(O)-Ar-L-D aryl hydrazide;

(Q)-NH—NH-L-D hydrazine;

(Q)-NH—NH-Ar-L-D aryl hydrazine;
(Ar) represents optionally substituted aryl groups including, but not limited to, optionally substituted phenyl, optionally substituted benzyl and optionally substituted benzoyl.

In one embodiment, taking into account the relevant chemistry of the acyl acceptors, the resulting molecules comprises an optionally substituted moiety selected from:

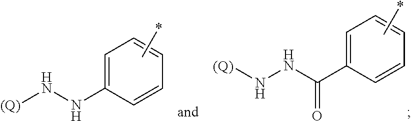

in particular

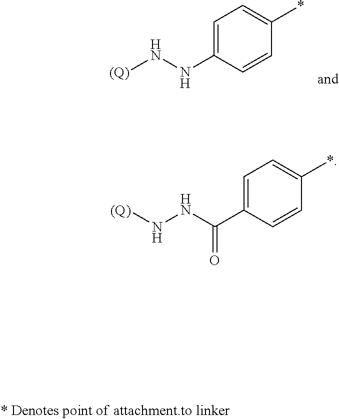

Alternatively, the acyl acceptor comprises an optionally substituted moiety selected from:

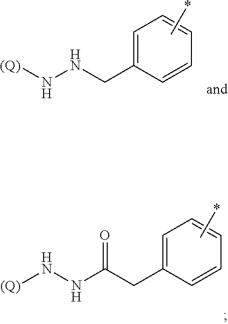

in particular

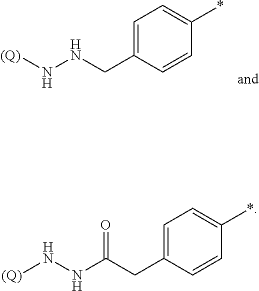

In a further embodiment, the acyl acceptor comprises an optionally substituted moiety selected from

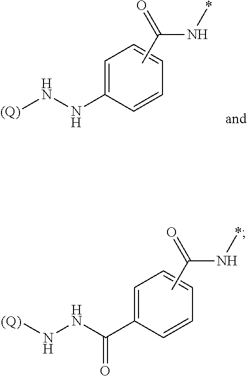

in particular

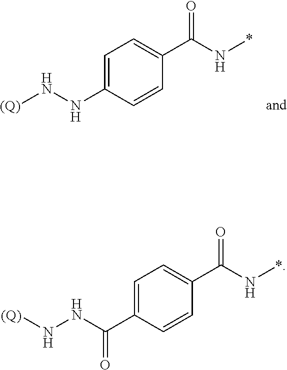

In a further alternative embodiment, the acyl acceptor comprises a moiety selected from

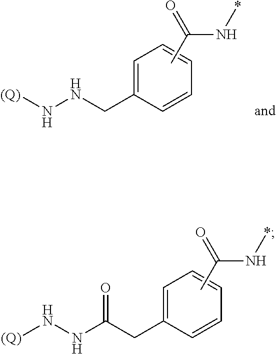

in particular

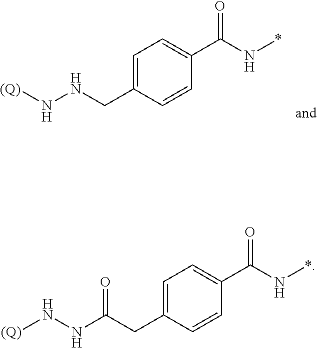

In one embodiment, the aryl derivative may be multiply substituted such that the acyl acceptor is branched with multiple points of attachment.

In particular embodiments, taking into account the relevant chemistry of the acyl acceptors, the resulting molecules may comprise any one of the following formulae:

(Q)-NH-Ar-(L-D)_{y} aryl amine;

(Q)-NH—O-Ar-(L-D)_{y} aryl aminooxy;

(Q)-NH—NH—C(O)-Ar-(L-D)_{y} aryl hydrazide;

(Q)-NH—NH-Ar-(L-D)_{y} aryl hydrazine;
wherein y is 2, 3, 4 or 5, in particular 2, 3, or 4, in particular 2 or 3, most particularly 2.

L and D in each occurrence, may be the same or different.

Alternatively it may be convenient to use a two-step procedure whereby an acyl acceptor moiety includes a reactive group (R), that enables the remainder of the moiety of interest, such as the drug-linker, to be added in a second step based on a further reaction with the reactive group. In such an embodiment, the remainder of the moiety of interest comprises a complementary reactive group R′ which can react with R.

For example, in one embodiment R or R′ is a moiety comprising a bioorthogonal-reaction compatible reactive group, for example an unprotected or protected thiol, epoxide, maleimide, haloacetamide, o-phoshenearomatic ester, azide, fulminate, sulfonate ester, alkyne, cyanide, amino-thiol, carbonyl, aldehyde, generally any group capable of oxime and hydrazine formation, 1,2,4,5-tetrazine, norbornene, other stained or otherwise electronically activated alkene, a substituted or unsubstituted cycloalkyne, generally any reactive groups which form via bioorthogonal cycloaddition reaction a 1,3- or 1,5-disubstituted triazole, any diene or strained alkene dienophile that can react via inverse electron demand Diels-Alder (IEDDA) reaction, a protected or unprotected amine, a carboxylic acid, an aldehyde, an oxyamine.

R or R′ can for example chosen to undergo thio-maleimide (or haloacetamide) addition, Staudinger ligation, Huisgen 1,3-cycloaddition (click reaction), or Diels-Alder cycloaddition with the complementary reactive group R′ to the remainder attached to of the moiety of interest, such as a drug-linker.

In one embodiment, the reactive group is a haloacetamide, (e.g. bromo-acetamide, iodo-acetamide, chloro-acetamide). Such reactive groups will be more stable in vivo (and in serum) compared with maleimide groups. In one embodiment, the reactive group is a reagent capable of undergoing a “click” reaction. For example a 1,3-dipole-functional compound can react with an alkyne in a cyclization reaction to form a heterocyclic compound, preferably in the substantial absence of added catalyst (e.g., Cu(I)). A variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

Examples include o-phosphenearomatic ester, an azide, a fulminate, an alkyne (including any strained cycloalkyne), a cyanide, an anthracene, a 1,2,4,5-tetrazine, or a norbornene (or other strained cycloalkene).

In one embodiment, R or R′ is a group having a terminal alkyne or azide; such moieties are described for example in U.S. Pat. No. 7,763,736.

In one embodiment, R or R′ is a substituted or unsubstituted cycloalkyne, such as a substituted or unsubstituted heterocyclic strained alkyne. For example R or R′ may be a DBCO (dibenzycyclooctyl) group or a BCN (bicyclononyne) group.

Alkynes such as those described herein above can be reacted with at least one 1,3-dipole-functional compound in a cyclization reaction to form a heterocyclic compound, preferably in the substantial absence of added catalyst (e.g., Cu(I)). A wide variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

Thus, in one embodiment, after the first step of the process the resulting molecule has the following formula: (Q)-NH-L₁-R; wherein Q is a glutamine residue present in the Fc region of the polypeptide; NH is derived from the acyl acceptor; L₁ is an optional linker or spacer moiety and R is a reactive group as described above. This can then be reacted with an agent comprising R′, such as R′-L₂-D where L₂ is an optional linker or spacer moiety and D is a drug. Accordingly in this embodiment with respect to the resulting product, -L₁-R—R′-L₂- collectively form the linker, L, as described above. Therefore the resulting molecule has the following formula: (Q)-NH-L₁-R—R′-L₂-D; wherein Q is a glutamine residue present in the Fc region of the polypeptide; NH is derived from the acyl acceptor; L₁ and L₂ are independently absent, linker or spacer moieties (L₁-R—R′-L₂ collectively forming a linker, L, as described above), preferably at least one of L₁ and L₂ is not absent; and D is a drug moiety.

As for the single step process above, since different acyl acceptors may be used, particular embodiments include

(Q)-NH-L₁-R—R′-L₂-D;

(Q)-NH-Ar-L₁-R—R′-L₂-D;

(Q)-NH—O-L₁-R—R′-L₂-D;

(Q)-NH—O-Ar-L₁-R—R′-L₂-D;

(Q)-NH—NHC(O)-L₁-R—R′-L₂-D;

(Q)-NH—NHC(O)-Ar-L₁-R—R′-L₂-D;

(Q)-NH—NH-L₁-R—R′-L₂-D;

(Q)-NH—NH-Ar-L₁-R—R′-L₂-D.
(Ar) represents optionally substituted aryl groups including, but not limited to, optionally substituted phenyl, optionally substituted benzyl and optionally substituted benzoyl.

In particular embodiments, taking into account the relevant chemistry of the acyl acceptors, the resulting molecules may comprise any one of the following formulae:

(Q)-NH-Ar-(L₁-R—R′-L₂-D)_{y} aryl amine;

(Q)-NH—O-Ar-(L₁-R—R′-L₂-D)_{y} aryl aminooxy;

(Q)-NH—NH—C(O)-Ar-(L₁-R—R′-L₂-D)_{y} aryl hydrazide;

(Q)-NH—NH-Ar-(L₁-R—R′-L₂-D)_{y} aryl hydrazine;
wherein y is 2,3, 4, or 5, in particular 2, 3, or 4, in particular 2 or 3, most particularly 2.

L₁, R, R′ and D in each occurrence, may be the same or different.

With reference to the example of R and R′ given above, R—R′ can for example be an addition product of a thio-maleimide (or haloacetamide) addition, for example, a NS-disubstituted-3-thio-pyrrolidine-2,5-dione; Staudinger ligation, for example, a N,3- or N,4-substitued-5-diphenylphosphinoxide-benzoic amide; Huisgen 1,3-cycloaddition (click reaction), for example, a NS-disubstituted-3-thio-pyrrolidine-2,5-dione, 1,4-disubstituted-1,2,3-triazole, 3,5-disubstituted-isooxazole, or 3,5-disubstituted-tetrazole; Diels-Alder cycloaddition adduct, for example the 2,4-cycloaddition product between an O or N-substituted-5-norbornene-2-carboxylic ester or amide, N-substituted-5-norbornene-2,3-dicarboxylic imide, O or N-substituted-7-oxonorbornene-5-carboxylic ester or amide, or N-substituted-7-oxonorbornene-5,6-dicarboxylic imide and a 9-substituted anthracene or 3-substituted 1,2,4,5-tetrazine; inverse electron demand Diels-Alder cycloaddition adduct, for example the 2,4-cycloaddition product between a substituted cyclopropenyl and 3-substituted 1,2,4,5-tetrazine; or any high yield selective amidation or imidization reaction.

Examples of R—R′ include:

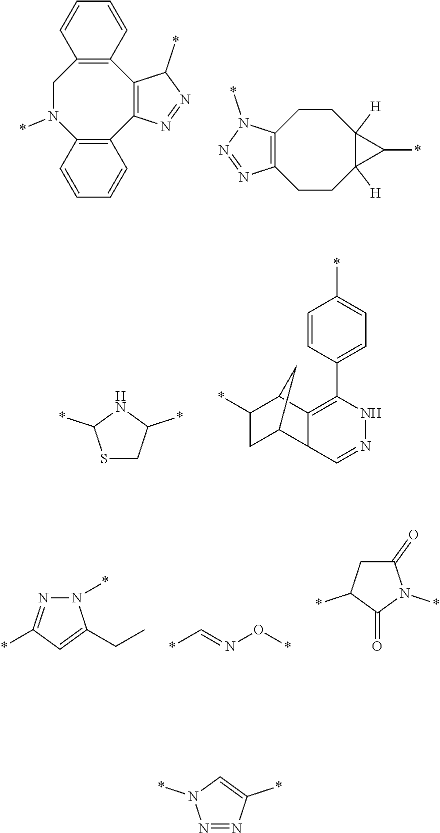

wherein (*) indicates the site of attachment of L₁ and L₂ or D. R—R′ can be in either orientation with respect to their attachment to L₁ and L₂ or D.

### Conjugation of a Second Drug-Linker at an Adjacent Remodeled N-Glycosylation Site

The N-glycosylation site, which has been treated with EndoS to trim it back to core GlcNAc, can be used for a second conjugation.

In one embodiment, this process comprises using enzymatic addition to the core GlcNAc moiety of a sugar derivative linked to a reactive group R (see previous section for examples of R), such as azide. Galactosyltransferases are a family of enzymes that catalyze the addition of a galactose residue from activated sugar nucleotide donor UDP-Gal to an acceptor in α1-3-, α1-4-, β1-3-, or β1-4-linkages. In one particular embodiment the enzyme is a β-1-4-galactosyltransferase (GalT), such as mutant version with a Y298L mutation (Ramakrishnan and Qasba, 2002, The Journal of Biological Chemistry 277 (23): 20833-20839). Other GalT mutants of interest include GalT Y289N, GalT Y2891, GalT Y289F, GalT Y289M, GalT Y289V, GalT Y289G, GalT Y2891 and GalT Y289A.

The general term “sugar” is herein used to indicate a monosaccharide, for example glucose (Glc), galactose (Gal), mannose (Man) and fucose (Fuc). The term “sugar derivative” is herein used to indicate a derivative of a monosaccharide sugar, i.e. a monosaccharide sugar comprising substituents and/or functional groups.

In one embodiment the sugar derivative together with the reactive group R is a sugar derivative Su(A)x-P derived from a sugar or a sugar derivative Su comprising x functional groups A, and P is a nucleoside mono- or diphosphate (P being part of the sugar derivative substrate for the enzymatic reaction but which is consumed during the reaction to leave the sugar derivative Su(A)x joined to the core GlcNAc residues). The sugar derivative Su(A)x comprises one or more functional groups A. When Su(A)x comprises two or more functional groups A, each functional group A is independently selected, i.e. one Su(A)x may comprise different functional groups A. In one embodiment, A is independently selected from the group consisting of an azido group, a keto group and an alkynyl group, and wherein x is 1, 2, 3 or 4; and P is a nucleoside mono- or diphosphate, such as selected from the group consisting of uridine diphosphate (UDP), guanosine diphosphate (GDP) and cytidine diphosphate (CDP). In a further embodiment, sugar derivative Su(A)x is preferably derived from galactose (Gal), mannose (Man), N-acetylglucosamine (GlcNAc), glucose (Glc), N-acetylgalactosamine (GalNAc), glucuronic acid (Gcu), fucose (Fuc) and N-acetylneuraminic acid (sialic acid), in particular from the group consisting of GlcNAc, Glc, Gal and GalNAc. In a yet further embodiment, Su(A)x is derived from Gal or GalNAc, in particular Su(A)x is derived from GalNAc.

In one embodiment, the one or more functional groups A in Su(A)x are present on C2 and/or C6 of the sugar or sugar derivative Su.

When A is an azido group, A is typically bonded to C2, C3, C4 or C6. As was described above, the one or more azide substituents in Su(A)x may be bonded to C2, C3, C4 or C6 of the sugar or sugar derivative S, instead of a hydroxyl (OH) group or, in case of 6-azidofucose (6-AzFuc), instead of a hydrogen atom. Alternatively or additionally, the N-acetyl substituent of an amino sugar derivative may be substituted by an azidoacetyl substituent. In one embodiment Su(A)x is selected from the group consisting of 2-azidoacetamidogalactose (GalNAz), 6-azido-6-deoxygalactose (6-AzGal), 6-azido-6-deoxy-2-acetamidogalactose (6-AzGalNAc), 4-azido-4-deoxy-2-acetamidogalactose (4-AzGalNAc), 6-azido-6-deoxy-2-azidoacetamidogalactose (6-AzGalNAz), 2-azidoacetamidoglucose (GlcNAz), 6-azido-6-deoxyglucose (6-AzGlc), 6-azido-6-deoxy-2-acetamidoglucose (6-AzGlcNAc), 4-azido-4-deoxy-2-acetamidoglucose (4-AzGlcNAc) and 6-azido-6-deoxy-2-azidoacetamidoglucose (6-AzGlcNAz), particularly from the group consisting of GalNAz, 6-AzGal, 6-AzGalNAc, 4-AzGalNAc, GlcNAz and 6-AzGlcNAc.

When A is a keto group, typically A is bonded to C2 instead of the OH group of Su. Alternatively A may be bonded to the N-atom of an amino sugar derivative, preferably a 2-amino sugar derivative. The sugar derivative then comprises an —NC(O)R³ substituent. R³ is preferably a C₂-C₄ alkyl group, optionally substituted.

In one embodiment, R³ is an ethyl group. In a further embodiment Su(A) is selected from the group consisting of 2-deoxy-(2-oxopropyl)galactose (2-ketoGal), 2-Npropionylgalactosamine (2-N-propionylGalNAc), 2-N-(4-oxopentanoyl)galactosamine (2-N-LevGal) and 2-N-butyrylgalactosamine (2-N-butyrylGalNAc), more preferably 2-ketoGalNAc and 2-N-propionyIGalNAc.

When A is an alkynyl group, in particular a terminal alkynyl group or a (hetero)cycloalkynyl group, typically said alkynyl group is present on a 2-amino sugar derivative. An example of Su(A)x wherein A is an alkynyl group is 2-(but-3-yonic acid amido)-2-deoxy-galactose.

In a particular embodiment, Su(A)x-P is selected from the group consisting of GalNAz-UDP 6-AzGal-UDP, 6-AzGalNAc-UDP, 4-AzGalNAz-UDP, 6-AzGalNAz-UDP, 6-AzGlc-UDP, 6-AzGlcNAz-UDP, 2-ketoGal-UDP, 2-N-propionyl-GalNAc-UDP and 2-(but-3-yonic acid amido)-2-deoxy-galactose-UDP. Most particularly, S(A)x-P is GalNAz-UDP, 4-AzGalNAc-UDP or 6-AzGalNAc-UDP.

Several compounds of the formula Su(A)x-P, wherein a nucleoside monophosphate or a nucleoside diphosphate P is linked to a sugar derivative Su(A)x, are known in the art. For example Wang et al, Chem. Eur. J. 2010, 16, 13343-13345, Piller et al., ACS Chem. Biol. 2012, 7, 753, Piller et al, Bioorg. Med. Chem. Lett. 2005,15, 5459-5462, WO 2009/102820, WO2014/065661, WO2015/057063, WO2015/057065, and WO2021/015622, all incorporated by reference herein, disclose a number of compounds Su(A)x-P and their syntheses.

Suitable conditions for the reaction of the core GlcNAc moiety with a sugar derivative comprising a reactive group in the presence of GalT are described in, for example, EP2,911,699, WO2016/022027; and WO2016/170186, all incorporated by reference herein.

In one embodiment wherein the process comprises using enzymatic addition to the core GlcNAc moiety of a sugar derivative linked to a reactive group R and wherein the sugar derivative together with the reactive group R is the sugar derivative Su(A)x-P as defined hereinabove, the resulting Fc-containing polypeptide, such as an antibody, comprises one or more of the following: (N)-G-S—R wherein N is an asparagine residue present in the Fc region of the polypeptide; G is an optionally fucosylated, GlcNAc N-glycan core; S is a sugar derivative; and R is the reactive group wherein S—R is the sugar derivative Su(A)x as defined hereinabove. A skilled person will appreciate that in this context, the nucleoside mono- or diphosphate P is not retained in the resulting Fc-containing polypeptide, such as an antibody, comprising a reactive group.

The resulting Fc-containing polypeptide, such as an antibody, comprising a reactive group, such as an azide group can then be conjugated to the second moiety of interest comprising an R′ reactive group, such as a drug-linker (e.g. as described above the two-step process for the first drug linker).

In one embodiment, the resulting Fc-containing polypeptide conjugate, such as an antibody conjugate, comprises one or more of the following: (N)-G-S—R—R′-L-D, or a pharmaceutically acceptable salt or solvate thereof; wherein N is an asparagine residue present in the Fc region of the polypeptide; G is an optionally fucosylated, GlcNAc N-glycan core; S is a sugar derivative; R—R′ is the reaction product between a reactive group R and a complementary reactive group R′; L is a linker, and D is a drug moiety.

To distinguish between the linker attached during the first conjugation and that attached during the second conjugation, the former may be referred to as LA and the latter LB.

In one embodiment, S—R is Su(A)x as defined above.

R—R′, L (LA and LB) and D are as defined above for the first step of the process where a glutamine residue is the site of conjugation. For the first conjugation D may be referred to as D1 and for the second conjugation D may be referred to as D2. D1 and D2 may be the same or different in the two conjugations i.e. the same to increase to the drug to antibody ratio (DAR) for a particular drug, or different to allow for simultaneously targeting of two different drugs to the same cell, such as drugs have a different mode of action (e.g. tubulin inhibitor and DNA binding agent). In a preferred embodiment, R—R′ for the second conjugation will typically be selected to be different to R—R′ for the first conjugation reaction to avoid any cross reactions i.e. in the first conjugation at the glutamine residue, R—R′ is R₁—R₁′; and in the second conjugation at asparagine-GlcNAc, R—R′ is R₂—R₂′; and R₁ does not react with R₂′ and R₂ does not react with R₁′.

Thus, in combination with the first process for addition of a moiety of interest at a glutamine residue, in one embodiment the Fc-containing polypeptide conjugate, such as an antibody conjugate, comprises:
(i) (Q)-NH-LA-D1, or a pharmaceutically acceptable salt or solvate thereof; wherein Q is a glutamine residue present in the Fc region of the polypeptide; LA is a linker; and D is a drug moiety; and
(ii) (N)-G-S—R—R′-LB-D2, or a pharmaceutically acceptable salt or solvate thereof; wherein N is an asparagine residue present in the Fc region of the polypeptide; G is an optionally fucosylated, GlcNAc N glycan core; S is a sugar derivative; R—R′ is the reaction product between a reactive group R and a complementary reactive group R′; LB is a linker; and D is a drug moiety; and
   wherein the asparagine residue is adjacent the glutamine residue.

For (i), all acyl acceptor variants described above in relation to the TGase reaction are included.

Where two sites are used, one based on using TGase at a glutamine residue and the other based on a GalT-mediated reaction at asparagine-GlcNAc site, it is not necessary to attach the complete first moiety of interest construct prior to the GalT-mediated reaction. For example, the TGase may be used to attach the acyl acceptor-reactive moiety (R), and then the sugar derivative and second reactive R group can be attached to the asparagine-GlcNAc N glycan core using a GalT-mediated reaction. If the R groups and R′ groups for the two sites are chosen to be different and not cross-reactive then, the first and second moieties of interest can then be added in a “one-pot” reaction at the same time, or sequentially if preferred.

Thus the following intermediate may be produced, which is an Fc-containing polypeptide, such as an antibody, comprising:
(i) (Q)-NH-L₁-R₁, wherein Q is a glutamine residue present in the Fc region of the polypeptide; L₁ is absent or a linker or spacer moiety, such as PEG; and R₁ is a reactive group; and
(ii) (N)-G-S—R₂, wherein N is an asparagine residue present in the Fc region of the polypeptide; G is an optionally fucosylated, GlcNAc N glycan core; S is a sugar derivative; and R₂ is a reactive group;
   wherein the asparagine residue is adjacent the glutamine residue.

This intermediate can then be reacted with (i) R₁′-L₂-D1; and (ii) R₂′-L₂-D2, wherein R₁′ and R₂′ are complementary reactive groups that react with R₁ and R₂, respectively and specifically i.e. R₁ does not react with R₂′ and R₂ does not react with R₁′. As an example, R₁ may be tetrazine and R₁′ is trans-cyclooctene (TCO); R₂ may be azide and R₂′ is dibenzocyclooctyne (DBCO).

This intermediate can be reacted with (i) R₁′-L₂-D1; and (ii) R₂′-L₂-D2 either simultaneously or sequentially, in either order.

A “pharmaceutically acceptable salt” is intended to mean a salt of a free acid or base of a compound of the invention, that is non-toxic, biologically tolerable, or otherwise biologically suitable for administration to a subject. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002.

Examples of pharmaceutically acceptable salts are those that are pharmacologically effective and suitable for contact with the tissues of subjects without undue toxicity, irritation, or allergic response. A compound of the invention may possess a sufficiently acidic group, a sufficiently basic group, or both types of functional groups, and accordingly react with a number of inorganic or organic bases, and inorganic and organic acids, to form a pharmaceutically acceptable salt.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate, trifluoroacetate and trifluoromethylsulfonate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoromethylsulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Examples of pharmaceutically acceptable salts particularly include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

A compound of the invention, or pharmaceutically acceptable salt thereof may be obtained as a solvate. Solvates include those formed from the interaction or complexation of compounds of the invention with one or more solvents, either in solution or as a solid or crystalline form. In some embodiments, the solvent is water and then the solvates are hydrates.

### Pharmaceutical Compositions

The conjugates disclosed herein which comprise one more pharmaceutically active substances/drugs, will typically be subjected to one or more purification steps e.g. to remove unused reactants, and reaction by-products. Such purification techniques include one or more of affinity purification and size exclusion purification. The resulting purified conjugate can be then combined with one or more pharmaceutically acceptable carriers or diluents. These typically include one or more of a buffering agent to maintain the pH at a desired level, such as from pH5 to pH7; a detergent such as polysorbate; and, especially in the case of lyophilized formulations, a lyoprotectant such as a sugar e.g. sucrose.

The pharmaceutical compositions may be used to treat a disease or disorder in an animal or human subject, such as a proliferative disorder e.g. cancer.

### Specific Embodiments

[1]. A method for conjugating a moiety of interest to an Fc-containing polypeptide (e.g. an antibody), which method comprises:
   (i) providing an Fc-containing polypeptide with N-linked glycosylation at a site present in the Fc region of the polypeptide, adjacent a site comprising a glutamine residue;
   (ii) treating said polypeptide with an enzyme to trim back the N-linked glycans at the site of N-linked glycosylation to an optionally fucosylated, core N-acetylglucosamine (GlcNAc) moiety; and
   (iii)(1) reacting, in the presence of a transglutaminase (TGase), the polypeptide obtained in step (ii) with a moiety of interest comprising an acceptor moiety which is a substrate for the TGase, so that the moiety of interest is attached to the polypeptide via the glutamine residue.
[2]. A method for conjugating a moiety of interest to an Fc-containing polypeptide (e.g. an antibody), which method comprises:
   (i) providing an Fc-containing polypeptide with N-linked glycosylation at a site present in the Fc region of the polypeptide, adjacent a site comprising a glutamine residue;
   (ii) treating said polypeptide with an enzyme to trim back the N-linked glycans at the site of N-linked glycosylation to an optionally fucosylated, core N-acetylglucosamine (GlcNAc) moiety; and
   (iii)(2) reacting, in the presence of a transglutaminase (TGase), the polypeptide obtained in step (ii) with an acceptor moiety which is a substrate for the TGase and which acceptor moiety further comprises a reactive group (R) which enables the subsequent attachment of the moiety of interest via a reaction between R and a complementary reactive group (R′) attached to the moiety of interest, such that the acceptor moiety is attached to the polypeptide via the glutamine residue; and
   (iv) reacting the polypeptide conjugate formed in step (iii)(2) with the moiety of interest attached to R′ under conditions such that R reacts with R′ to form R—R′.
[3]. A method according to [1] or [2] wherein the acceptor moiety is selected from an amine, an aminooxy, a hydrazido, a hydrazino and aryl derivatives thereof.
[4]. A method according to [2] or [3] wherein the acceptor moiety is of formula Ac-Sp-R where Ac is selected from an amine, an aminooxy, a hydrazido, a hydrazino and aryl derivatives thereof, Sp is absent or a spacer moiety; and R is a reactive group, e.g. an azide.
[5]. A method according to any one of the preceding embodiments wherein the enzyme is EndoS.
[6]. A method according to any one of the preceding embodiments wherein the N-linked glycosylation site is at position 297 according to the Kabat numbering system, or an equivalent position and/or the site comprising a glutamine residue is at position 295 according to the Kabat numbering system, or an equivalent position.
[7]. A method according to any one of the preceding embodiments wherein the moiety of interest comprises a drug-linker.
[8]. A method according to any one of the preceding embodiments wherein a second moiety of interest is conjugated to the Fc-containing polypeptide, which method further comprises the following step:
   (v) reacting, in the presence of a galactosyl transferase (GalT), the polypeptide obtained in step (iii)(1), step (iii)(2) or step (iv) with a sugar derivative attached to the second moiety of interest, so that the sugar derivative is attached to the polypeptide via the core N-acetylglucosamine (GlcNAc) moiety.
[9]. A method according to any one of embodiments [1] to [7] wherein a second moiety of interest is conjugated to the Fc-containing polypeptide, which method further comprises the following steps:
   (v) reacting, in the presence of a galactosyl transferase (GalT), the polypeptide obtained in step (iii))(1), step (iii)(2) or step (iv) with a sugar derivative attached to a reactive group (R) which enables the subsequent attachment of the moiety of interest via a reaction between R and a complementary reactive group (R′) attached to the moiety of interest, so that the sugar derivative is attached to the polypeptide via the core N-acetylglucosamine (GlcNAc) moiety; and
   (vi) reacting the polypeptide conjugate formed in step (v) with the moiety of interest attached to R′ under conditions such that R reacts with R′ to form R—R′.
[10]. An Fc-containing polypeptide conjugate, which comprises:
   (i) an asparagine residue present in the Fc region of the polypeptide with an optionally fucosylated, GlcNAc N-glycan core lacking any additional endogenous glycan residues; and
   (ii) (Q)-NH-L-D, or a pharmaceutically acceptable salt or solvate thereof; wherein Q is a glutamine residue present in the Fc region of the polypeptide; L is a linker, which optionally comprises a conditionally-cleavable moiety; and D is a drug moiety;
   wherein the asparagine residue is adjacent the glutamine residue.
[11]. An Fc-containing polypeptide conjugate according to [10], wherein L comprises L₁-R—R′-L₂;
   wherein L₁ is absent or a spacer moiety, R—R′ is formed by the reaction of a reactive group R and a complementary reactive group R′; and L₂ is a linker or spacer moiety to which D is attached and which optionally comprises a conditionally-cleavable moiety.
[12]. An Fc-containing polypeptide conjugate, which comprises:
   (i) (Q)-NH-LA-D1, or a pharmaceutically acceptable salt or solvate thereof; wherein Q is a glutamine residue present in the Fc region of the polypeptide; LA is a linker; and D is a drug moiety;
   (ii) (N)-G-S—R₂—R₂′-LB-D2, or a pharmaceutically acceptable salt or solvate thereof; where N is an asparagine residue present in the Fc region of the polypeptide; G is an optionally fucosylated, GlcNAc N-glycan core; S is a sugar derivative; R₂—R₂′ is formed by the reaction of a reactive group R₂ and a complementary reactive group R₂′; LB is a linker, which optionally comprises a conditionally-cleavable moiety; and D1 and D2 are each independently a drug moiety; and
   wherein the asparagine residue is adjacent the glutamine residue.
[13]. An Fc-containing polypeptide conjugate according to [12], wherein LA comprises LA₁-R₁—R₁′-LA₂;
   wherein LA₁ is absent or a spacer moiety, R₁—R₁′ is formed by the reaction of a reactive group R₁ and a complementary reactive group R₁′; and LA₂ is a linker or spacer moiety to which D1 is attached and which optionally comprise a conditionally-cleavable moiety.
[14]. An Fc-containing polypeptide conjugate according to any one of to wherein D is a cytotoxin or immunostimulatory molecule.
[15]. An Fc-containing polypeptide conjugate according to any one of to wherein the asparagine residue is at position 297 according to the Kabat numbering system, or an equivalent position and/or the glutamine residue is at position 295 according to the Kabat numbering system, or an equivalent position.

Some aspects and embodiments of the disclosure are described below in more detail with reference to the following examples, which are illustrative only and non-limiting.

The examples refer to the following figures:

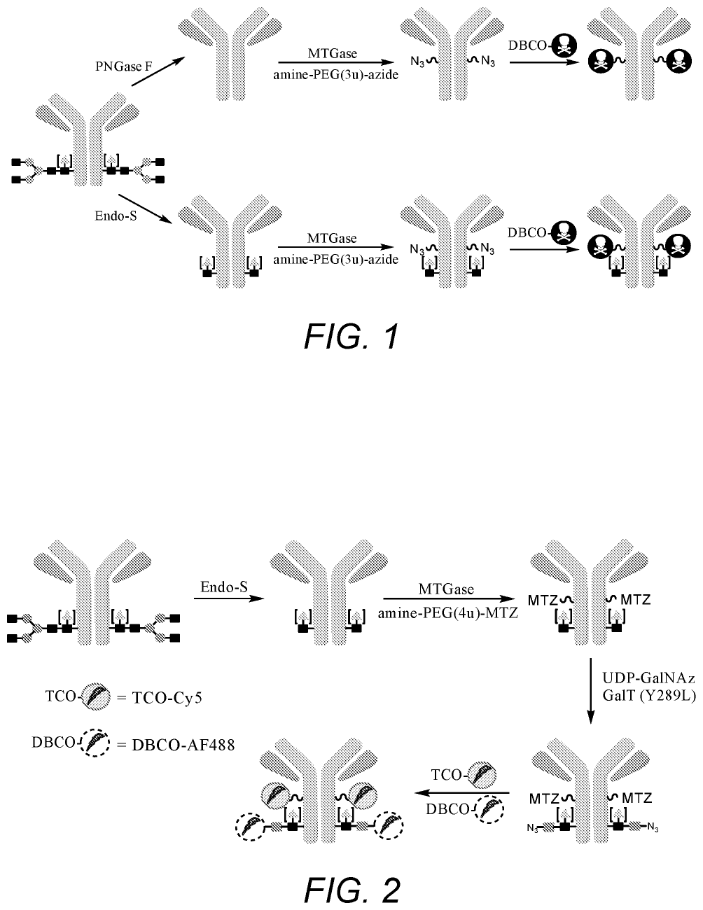

FIG. 1—MTGase mediated conjugation of amine-PEG (3u)-azide to an antibody degylcosylated using PNGase F (top) or glycan trimmed using EndoS (bottom).

FIG. 2—Dual-conjugation to an antibody functionalized with amine-PEG (4u)-MTZ via MTGase mediated conjugation and GalNAz via glycan remodelling. TCO-Cy5 and DBCO-AF488 were used to model the process, conjugating at MTZ and azide functional groups respectively.

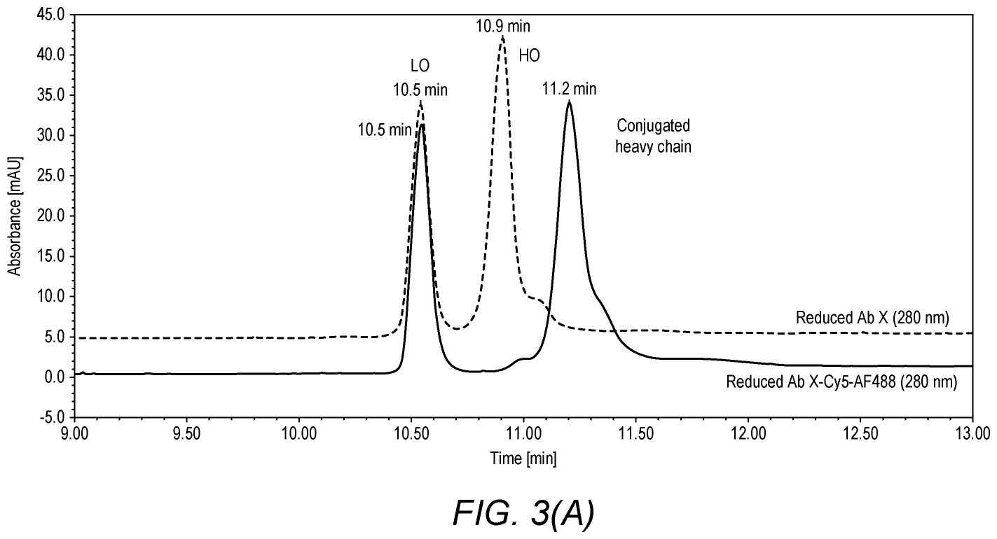

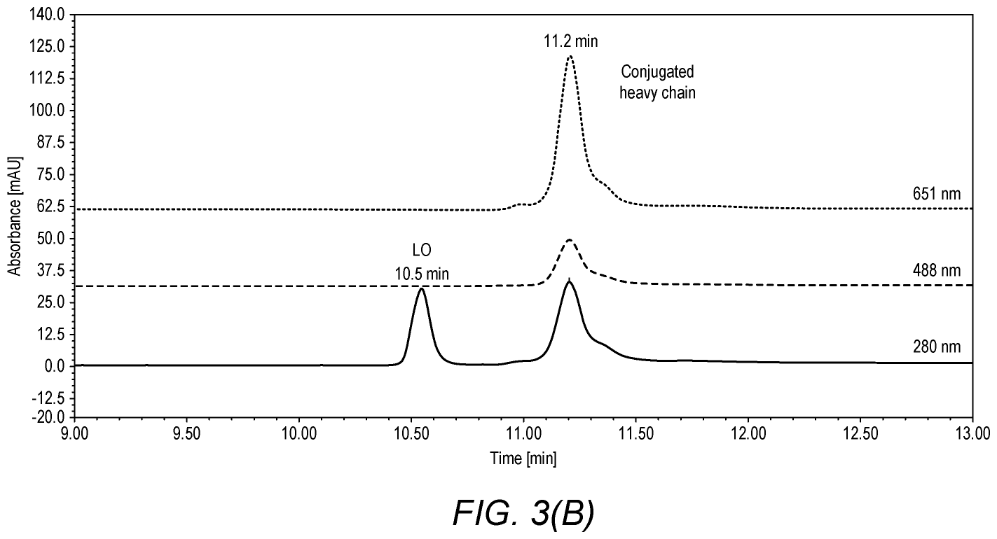

FIG. 3—(A) Reducing PLRP of antibody X and antibody X dual-labelled with TCO-Cy5 and DBCO-AF488; (B) Reducing PLRP of antibody X dual-labelled with TCO-Cy5 and DBCO-AF488 with absorbance maxima at 280 nm (protein), 488 nm (AF488) and 651 nm (Cy5) overlaid.

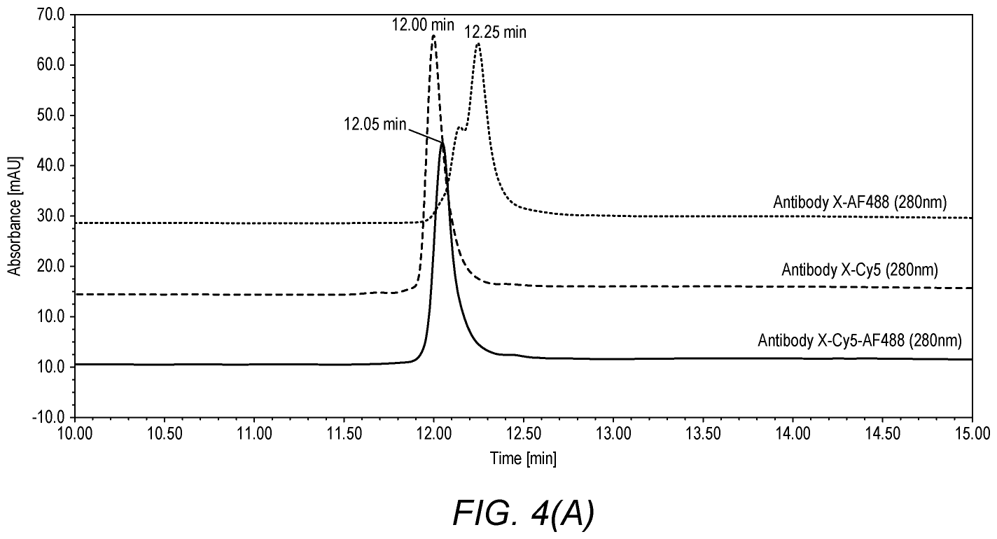

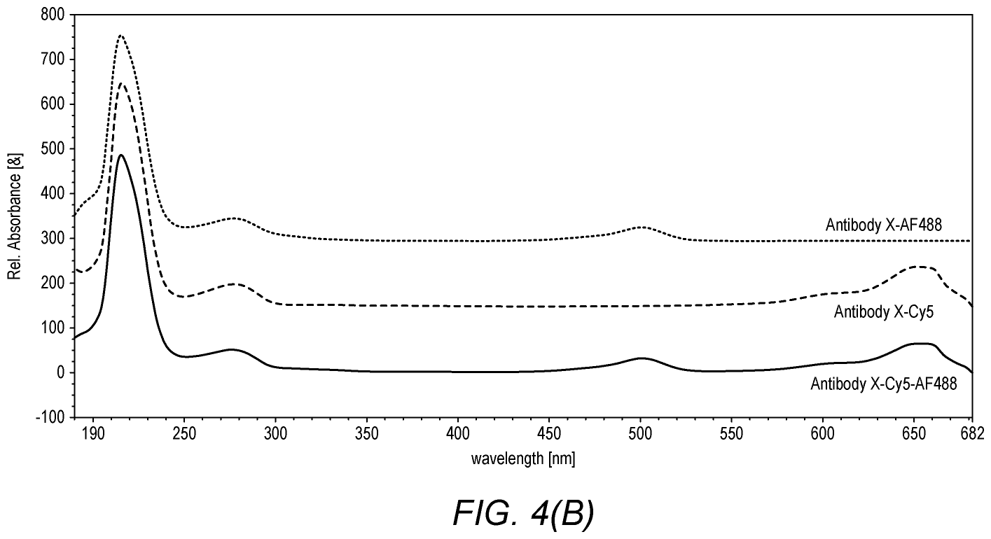

FIG. 4—(A) Non-reducing PLRP of dual and mono-labelled of antibody X labelled with TCO-Cy5 and DBCO-AF488; (B) UV/Vis spectra of dual and mono-labelled antibody X.

### EXAMPLES

### Example 1—EndoS Versus PNGase F Prior to MTGase Treatment

Transamidation reactions between primary amine donors and the side-chain of glutamine residue can be catalysed by microbial transglutaminase. This biochemistry has been exploited for the conjugation of cytotoxic drugs to antibodies. IgG has a conserved site for transglutaminase mediated conjugation at Q295. To ensure sufficient surface accessibility to Q295, prior to MTGase mediated conjugation, the antibody is typically deglycosylated at the proximal N297 residue using PNGase F. We have developed a new process, where the glycan at N297 is trimmed to the core GlcNAc[Fuc] using EndoS prior to MTGase conjugation of an azide-based acyl acceptor, amine-PEG (3u)-azide.

### Materials and Methods

### PNGase Deglycosylation of Antibody X

Antibody X (2.5 mg) was degylcosylated using PNGase F (379 KU) at an antibody concentration of 1.33 mg/mL, the digest was incubated at 37° C. for 18 h. Deglycosylated antibody X was purified by protein A chromatography followed by buffer exchange into PBS and concentration by UF/DF.

### EndoS Glycan Trimming of Antibody X

Antibody X (2.5 mg) glycans were trimmed using EndoS (12.5 μg) at an antibody concentration of 1.0 mg/mL, the digest was incubated at 37° C. for 18 h. Trimmed antibody X was purified by protein A chromatography followed by buffer exchange into PBS and concentration by UF/DF.

### MTGase Mediated Conjugation of Linkers 1-4, 7, 8 & 11

Solutions of linkers 1-4, 7, 8 & 11 (Table 1) were prepared in PBS. Degylcosylated/trimmed antibody X samples (0.1 mg, 2 mg/mL) were next functionalized with linkers 1-4, 7, 8 & 11 (200 eq.) via MTGase mediated conjugation using 6.7 U of enzyme per mg of antibody. The resulting reactions were incubated at 37° C. for 18 h. Functionalized antibody X samples were purified by protein A chromatography followed by buffer exchange into PBS and concentration via UF/DF. The efficiency of conjugation for the purified samples was determined by LC-MS (Table 2).

TABLE 1
Molecular structures of linkers 1-4, 7, 8 & 11

| | | |
|---|---|---|
| 1 | amine-PEG(3u)-azide | 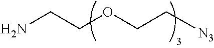 |
| 2 | aminooxy-PEG(3u)-azide | 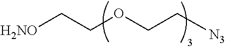 |
| 3 | hydrazido-PEG(4u)-azide | 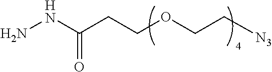 |
| 4 | hydrazino-PEG(4u)-azide | 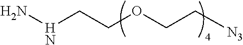 |
| 7 | 4-hydrazidobenzamide- PEG(4u)-azide | 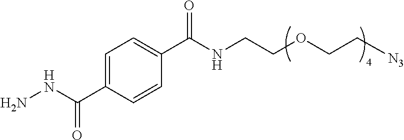 |
| 8 | 4-hydrazinobenzamide- PEG(4u)-azide | 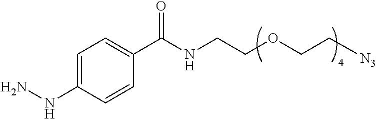 |
| 11 | 4-hydrazidomethyl- benzamide-PEG(4u)- azide | 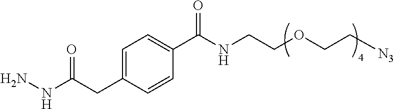 |

### Results

TABLE 2
Efficiency of MTGase mediated conjugation for various alpha-effect
nucleophile linkers using PNGase F and EndoS processes.

| | | PNGase F | EndoS |
|---|---|---|---|
| Compound | Linker | Process | Process |
| 1 | amine-PEG(3u)-azide | 100% | 100% |
| 2 | aminooxy-PEG(3u)-azide | 95% | 84% |
| 3 | hydrazido-PEG(4u)-azide | 98% | 97% |
| 4 | hydrazino-PEG(4u)-azide | 89% | 86% |
| 7 | 4-hydrazidobenzamide-PEG(4u)-azide | N.D. | 100% |
| 8 | 4-hydrazinobenzamide-PEG(4u)-azide | 91% | 84% |
| 11 | 4-hydrazidomethylbenzamide-PEG(4u)-azide | N.D. | 100% |

N.D.: Not determined

These results show that a process based on an EndoS process works well.

### Example 2—Conjugation of DBCO-PEG (5 kDa) and DBCO-Cy5 to Azide Functionalized Antibody X Samples

To model the conjugation process, functionalized antibody X samples from Example 1 were conjugated with DBCO-PEG (5 kDa) and DBCO-Cy5. Briefly, functionalized antibody X samples (50 μg) were incubated with either DBCO-PEG (5 kDa) and DBCO-Cy5 (10 eq.) at 0.5 mg/mL antibody concentration. The resulting reaction mixtures were incubated at 22° C. for 19 h. To assess the conjugation efficiency the DBCO-PEG (5 kDa) conjugations were analysed by SDS-PAGE directly. The DBCO-Cy5 conjugates were buffer exchanged into PBS via gel filtration and then assessed by LC-MS.

PEGylation of the heavy chain of Antibody X was observed by reducing SDS-PAGE with degrees of PEGylation of 1.3 and 1.5 determined for PNGase F and EndoS respectively. Cy5 conjugation was monitored by MS: The PNGase F process was completed with 74% efficiency at the DBCO-Cy5 conjugation step (DoL=1.5); The EndoS process was efficient with 94% incorporation of the DBCO-Cy5 observed (DoL=1.9).

The EndoS process was therefore observed to be over 25% more efficient than the PNGase F process.

### Example 3—Dual Labelling at Q295/N297 with MTGase and Galactosyltransferase

The single congution approach in Examples 1 and 2 was combined with a second conjugation at the EndoS trimmed site at N297.

### Materials and Methods

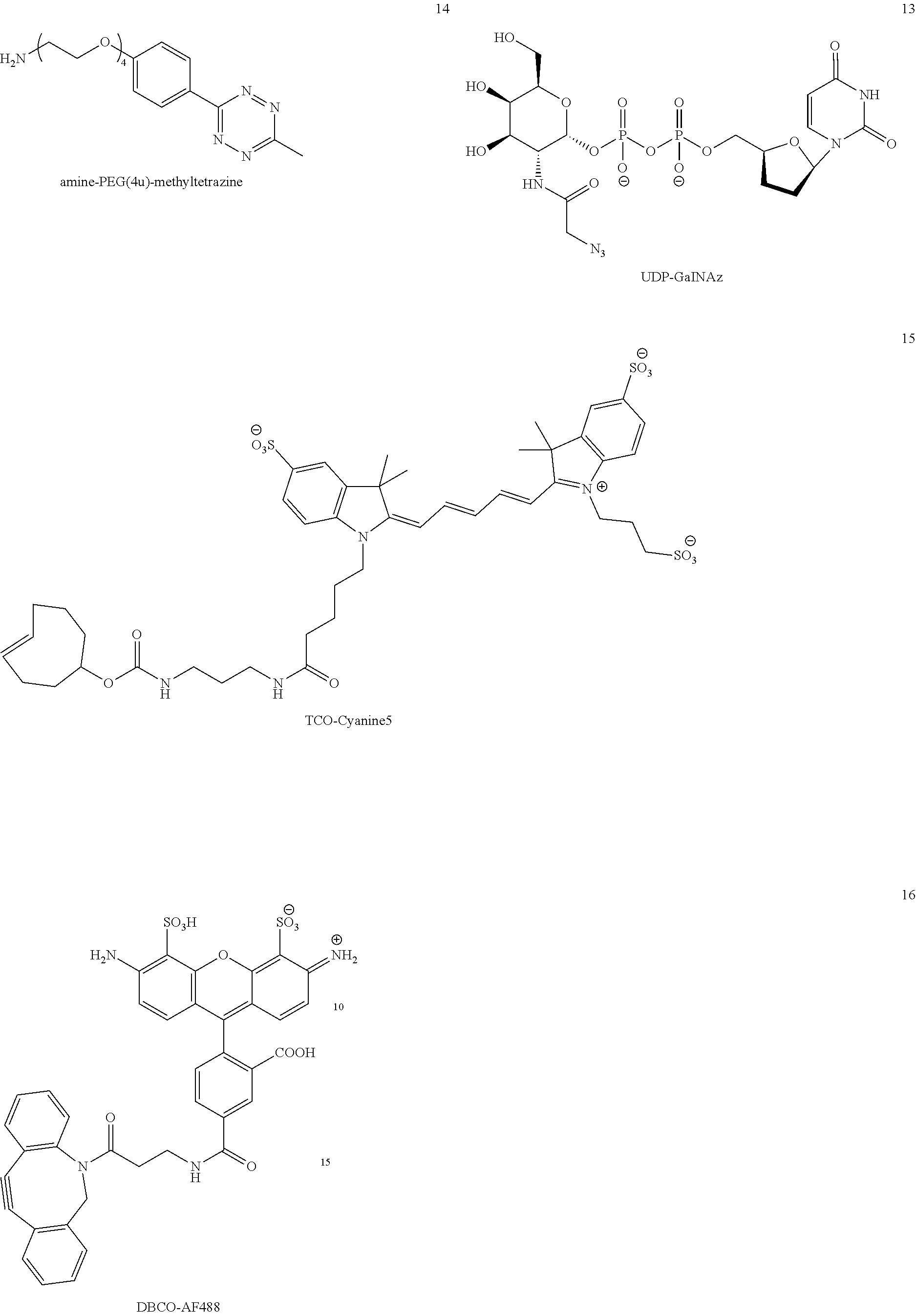

EndoS Glycan Trimming and MTGase Mediated Conjugation of Amine-PEG (4u)-MTZ of Antibody X

Antibody X glycans were trimmed at 1 mg scale using EndoS as per Example 1. Next, trimmed antibody X (0.65 mg) was functionalized at 0.8 mg/mL antibody concentration with amine-PEG (4u)-MTZ (80 eq.) via MTGase mediated conjugation using 6 U/mL of enzyme. The reaction was incubated at 37° C. overnight. Purification of conjugate was performed by protein A chromatography followed by buffer exchange into PBS and concentration via UF/DF.

### Glycan Remodelling for Mono-Functionalized Antibody X

Mono-functionalized antibody X (0.5 mg) was remodelled via GalT (Y289L) transfer of UDP-GalNAz using the SiteClick™ conjugation kit as per the manufacturer's instructions. Remodelling was performed at 30° C. overnight. Purification of dual functionalized antibody X was performed by UF/DF, buffer exchanging into TBS, pH 7.

Conjugation of TCO-Cy5 and/or DBCO-AF488 to Dual Functionalized Antibody X

Solutions of TCO-Cy5 and DBCO-AF488 in DMSO were prepared at 10 mM concentration. The dual functionalized antibody X (0.1 mg) was labelled with TCO-Cy5 (10 eq.) and DBCO-AF488 (10 eq.) at 4 mg/mL antibody concentration. Control reactions where only one fluorophore was added were also performed. The conjugation reactions were incubated at 22° C. overnight. Purification of conjugates was performed by protein A chromatography followed by buffer exchange into PBS and concentration via UF/DF. The purified conjugates were analysed by PLRP and LC-MS.

### Results

Conjugation was confirmed by a shift in the retention time of the heavy chain of the conjugate compared to the naked antibody using RP-UHPLC (Vanquish™). Absorbance at 651 nm (Cy5) and 488 nm (AF488) in the heavy chain confirmed conjugation with both fluorophores (FIG. 3).

PLRP of the dual-labelled conjugate and mono-labelled samples with UV/Vis spectral analysis clearly show the dual labelled conjugate to contain both fluorophores with the mono-labelled conjugates only showing single absorbance maxima for their respective fluorophores (FIG. 4).

MS analysis indicated that the incorporation level for amine-PEG (4u)-methyltetrazine was 90%. Glycan remodelling with GalNAz gave complete reaction and the conjugation of TCO-Cy5 and DBCO-AF488 to their respective sites also went to complete conversion.

### CONCLUSION

We have shown that, using a combination of two conjugation techniques, we can use both the Q295 and N297 sites in the same antibody to dual label antibodies efficiently. This dual labelling method can be used create nearly homogenous antibody drug conjugates (ADCs) with two different cytotoxins attached to one antibody. Two mechanisms of action could improve treatment outcomes and reduce drug resistance as many systemic cancer treatments use a combination of drugs. The homogeneity of ADCs has been shown to increase efficacy and safety as suboptimal conjugate species deliver too little payload (low DAR variants) or potentially have suboptimal pharmacokinetics (high DAR variants).

A number of publications are cited above to more fully describe and disclose the disclosures and the state of the art to which inventions herein may pertain. The entirety of each of the references mentioned in this disclosure are hereby is incorporated by reference.

1. A method for conjugating a moiety of interest to an Fc-containing polypeptide (e.g. an antibody), which method comprises:
(i) providing an Fc-containing polypeptide with N-linked glycosylation at a site present in the Fc region of the polypeptide, adjacent a site comprising a glutamine residue;
(ii) treating said polypeptide with an enzyme to trim back the N-linked glycans at the site of N-linked glycosylation to an optionally fucosylated, core N-acetylglucosamine (GlcNAc) moiety; and
(iii)(1) reacting, in the presence of a transglutaminase (TGase), the polypeptide obtained in step (ii) with a moiety of interest comprising an acceptor moiety which is a substrate for the TGase, so that the moiety of interest is attached to the polypeptide via the glutamine residue.

2. A method for conjugating a moiety of interest to an Fc-containing polypeptide (e.g. an antibody), which method comprises:
(i) providing an Fc-containing polypeptide with N-linked glycosylation at a site present in the Fc region of the polypeptide, adjacent a site comprising a glutamine residue;
(ii) treating said polypeptide with an enzyme to trim back the N-linked glycans at the site of N-linked glycosylation to an optionally fucosylated, core N-acetylglucosamine (GlcNAc) moiety; and
(iii)(2) reacting, in the presence of a transglutaminase (TGase), the polypeptide obtained in step (ii) with an acceptor moiety which is a substrate for the TGase and which acceptor moiety further comprises a reactive group (R) which enables the subsequent attachment of the moiety of interest via a reaction between R and a complementary reactive group (R′) attached to the moiety of interest, such that the acceptor moiety is attached to the polypeptide via the glutamine residue; and
(iv) reacting the polypeptide conjugate formed in step (iii)(2) with the moiety of interest attached to R′ under conditions such that R reacts with R′ to form R—R′.

3. A method according to claim 1 or claim 2 wherein the acceptor moiety is selected from an amine, an aminooxy, a hydrazido, a hydrazino and aryl derivatives thereof.

4. A method according to claim 2 or claim 3 wherein the acceptor moiety is of formula Ac-Sp-R where Ac is selected from an amine, an aminooxy, a hydrazido, a hydrazino and aryl derivatives thereof, Sp is absent or a spacer moiety; and R is a reactive group, e.g. an azide.

5. A method according to any one of the preceding claims wherein the enzyme is EndoS.

6. A method according to any one of the preceding claims wherein the N-linked glycosylation site is at position 297 according to the Kabat numbering system, or an equivalent position and/or the site comprising a glutamine residue is at position 295 according to the Kabat numbering system, or an equivalent position.

7. A method according to any one of the preceding claims wherein the moiety of interest comprises a drug-linker.

8. A method according to any one of the preceding claims wherein a second moiety of interest is conjugated to the Fc-containing polypeptide, which method further comprises the following step:
(v) reacting, in the presence of a galactosyl transferase (GalT), the polypeptide obtained in step (iii)(1), step (iii)(2) or step (iv) with a sugar derivative attached to the second moiety of interest, so that the sugar derivative is attached to the polypeptide via the core N-acetylglucosamine (GlcNAc) moiety.

9. A method according to any one of claims 1 to 7 wherein a second moiety of interest is conjugated to the Fc-containing polypeptide, which method further comprises the following steps:
(v) reacting, in the presence of a galactosyl transferase (GalT), the polypeptide obtained in step (iii))(1), step (iii)(2) or step (iv) with a sugar derivative attached to a reactive group (R) which enables the subsequent attachment of the moiety of interest via a reaction between R and a complementary reactive group (R′) attached to the moiety of interest, so that the sugar derivative is attached to the polypeptide via the core N-acetylglucosamine (GlcNAc) moiety; and
(vi) reacting the polypeptide conjugate formed in step (v) with the moiety of interest attached to R′ under conditions such that R reacts with R′ to form R—R′.

10. An Fc-containing polypeptide conjugate, which comprises:
(i) an asparagine residue present in the Fc region of the polypeptide with an optionally fucosylated, GlcNAc N-glycan core lacking any additional endogenous glycan residues; and
(ii) (Q)-NH-L-D, or a pharmaceutically acceptable salt or solvate thereof; wherein Q is a glutamine residue present in the Fc region of the polypeptide; L is a linker, which optionally comprises a conditionally-cleavable moiety; and D is a drug moiety;
wherein the asparagine residue is adjacent the glutamine residue.

11. An Fc-containing polypeptide conjugate according to claim 10, wherein L comprises L₁-R—R′-L₂;
wherein L₁ is absent or a spacer moiety, R—R′ is formed by the reaction of a reactive group R and a complementary reactive group R′; and L₂ is a linker or spacer moiety to which D is attached and which optionally comprises a conditionally-cleavable moiety.

12. An Fc-containing polypeptide conjugate, which comprises:
(i) (Q)-NH-LA-D1, or a pharmaceutically acceptable salt or solvate thereof; wherein Q is a glutamine residue present in the Fc region of the polypeptide; LA is a linker; and D is a drug moiety;
(ii) (N)-G-S—R₂—R₂′-LB-D2, or a pharmaceutically acceptable salt or solvate thereof; where N is an asparagine residue present in the Fc region of the polypeptide; G is an optionally fucosylated, GlcNAc N-glycan core; S is a sugar derivative; R₂—R₂′ is formed by the reaction of a reactive group R₂ and a complementary reactive group R₂′; LB is a linker, which optionally comprises a conditionally-cleavable moiety; and D1 and D2 are each independently a drug moiety; and
wherein the asparagine residue is adjacent the glutamine residue.

13. An Fc-containing polypeptide conjugate according to claim 12, wherein LA comprises LA₁-R₁—R₁′-LA₂;
wherein LA₁ is absent or a spacer moiety, R₁—R₁′ is formed by the reaction of a reactive group R₁ and a complementary reactive group R₁′; and LA₂ is a linker or spacer moiety to which D1 is attached and which optionally comprise a conditionally-cleavable moiety.

14. An Fc-containing polypeptide conjugate according to any one of claims 10 to 15 wherein D is a cytotoxin or immunostimulatory molecule.

15. An Fc-containing polypeptide conjugate according to any one of claims 10 to 14 wherein the asparagine residue is at position 297 according to the Kabat numbering system, or an equivalent position and/or the glutamine residue is at position 295 according to the Kabat numbering system, or an equivalent position.
